(19) United States
(12) Patent Application Publication
(10) Pub. No.: US 2022/0061430 A1
(43) Pub. Date: Mar. 3, 2022

(54) APPLICATOR GLOVE FOR DISPENSING A LIQUID OR GEL

(71) Applicant: KEDRICK EVANS, POTOMAC, MD (US)

(72) Inventor: KEDRICK EVANS, POTOMAC, MD (US)

(21) Appl. No.: 17/006,941

(22) Filed: Aug. 31, 2020

(51) Int. Cl. A41D 19/00

(52) U.S. Cl. CPC A41D 19/002

(57) ABSTRACT

An applicator glove to apply a liquid or gel having to a surface comprising a liquid permeable fabric sufficiently permeable to allow transfusion of said liquid such as body wash, detergent, sterilizer, disinfectant, lotion, oil, wax or other viscous substance. The glove includes a pocket defined in a palm positioned of said glove, a bladder positioned in the pocket, a fill port in the bladder to allow filling of a fluid reservoir where the walls of the bladder has slits or orifices to allow fluid passage when exceeding a threshold pressure generated by hand pressure.

### BACKGROUND

This invention concerns liquid or gel dispensing, but more specifically, to a glove or mitten useful for manually applying a liquid substance, such as a bathing liquid or gel during bathing, or alternatively, to a mitten or glove useful for manually applying a viscous liquid or gel-like substance to a surface while washing, disinfecting, sterilizing, waxing, lubricating or applying a coating.

Bath gloves are known in the prior art. Such gloves heretofore devised and utilized are known to consist basically of a pocket in the glove to hold a solid bar of soap that dissolves during bathing. Crowded prior art which have been developed for the fulfillment of countless objectives and requirements. Known prior art includes U.S. Pat. Nos. 6,398,443; 5,673,435; 3,885,249; 3,711,889; 4,183,100; U.S. Pat. No. Des. 309,603 and U.S. Pat. No. Des. 632,445.

While these devices fulfill their respective needs, this invention provides a means to dispense a viscous liquid or gel that may be used for a variety of purposes—not only bathing but also for waxing, disinfecting, sterilizing, lubricating, or applying by hand a substance to any type of surface.

### SUMMARY OF INVENTION

In accordance with an aspect of the present invention, there is provided an applicator glove useful for applying or dispensing a liquid or gel having a predetermined viscosity. The applicator glove generally comprises a fluid-permeable fabric wrapping to be worn on a hand of a user where the fabric is sufficiently permeable to allow transfusion of liquid or gel for application to a surface. The applicator glove may be used to treat a surface with a detergent, disinfectant, sterilizer, biocide, oil, wax, grease and other substance. The glove includes a pocket defined in the fabric positioned in or about a palm position thereof, a bladder positioned in the pocket where the bladder has an internal fluid reservoir surrounded by flexible walls and including plural longitudinal slits on an anterior portion thereof that enable passage of fluid from a reservoir to an exterior surface of the bladder. Instead of slits, the bladder may possess holes or orifices to dispense the liquid or gel. In the case of applying a toxic substance, the glove may in include an inner protective lining such as rubber or latex. The bladder also has a fill port to allow filling of the reservoir with liquid or gel where an opening of the fill port is biased in the closed position when the reservoir is filled with liquid or gel but is adapted to receive a puncture probe to fill the bladder with said liquid or gel. Biasing in the closed position may be accomplished by a check valve or other means known in the art. Further, the walls of the bladder has a thickness and rigidity to retain closure of the slits at less than a threshold pressure in the reservoir and to allow opening of said slit when the threshold pressure is exceeded. The threshold pressure in the reservoir is defined by hand pressure of a user and the viscosity of the liquid or gel when using said glove.

Other features of the invention include the applicator glove comprising either a natural and synthetic permeable fabric where the bladder comprises rubber or polyurethane. The bladder may have a wall thickness between one-eight and three-eight's of an inch, more or less.

Another aspect of the invention also includes a puncture probe to allow filing of the reservoir where the puncture probe is adapted at one end thereof for insertion into the bladder and, at the other end thereof, for insertion into a container of liquid or gel to be dispensed.

A further aspect of the invention comprises a tubular fluid conduit between distal ends of the puncture probe adapted for continuously supply fluid or gel to said bladder during use by a user. The applicator glove may take the form of a mitten or a finger-fit glove. Instead of slits, transfusion of liquid from the bladder may occur through orifices or holes that are sized according to the viscosity of the liquid or gel being dispensed.

In yet another aspect of the invention, there is provided a bladder having orifices of at least two difference sizes and at least one removable segment adapted to expose orifices of a different size according to user desire whereby to enable the bladder to dispense a liquid or gel of a different viscosity.

Other features, benefits and advantages of the invention will become readily apparent upon review of the following detailed description taken in connection with the accompanying drawings. The invention, though, is pointed out with particularity by the appended claims.

### BRIEF DESCRIPTION OF THE DRAWINGS

FIG. 1 depicts an applicator glove having a dispensing bladder in accordance with one aspect of the present invention.

FIG. 2 is a perspective view of the bladder of FIG. 1.

FIG. 3a is a cross-cut along line A-A of FIG. 2, which shows the thickness of the walls and open slits.

FIG. 3b shows a elongated puncture probe that may be used to fill the reservoir of the bladder of FIG. 1.

FIG. 3c show the wall of a container of liquid or gel, which may be body wash, an oil or grease, or other substance to be applied to a surface by the applicator glove of FIG. 1.

FIG. 4 show the applicator glove and bladder in relation to a user's hand.

FIG. 5 depicts a bladder having small holes or orifices (instead of slits), which may expand or contract according to internal pressure within the fluid reservoir of the bladder.

FIG. 6 show an applicator glove comprising a finger-fit glove instead of a mitten that may be used to apply a liquid or gel to surface, as well as a peel-away segment to expose different size orifices according to user selection.

### DETAIL DESCRIPTION OF AN EMBODIMENT

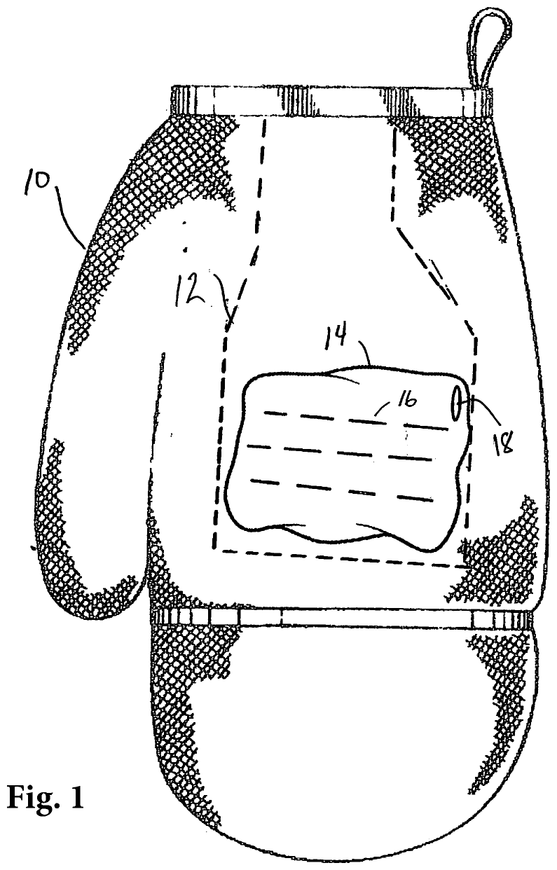

FIG. 1 depicts an applicator glove 10 according to a first aspect of the invention that is useful for applying or dispensing a liquid or gel such as a body wash detergent, disinfectant, sterilizer, or an oil, grease or any other viscous substance having a predetermined viscosity. Glove 10, which is shown here as a mitten but may also comprise a finger-fit glove, comprises a fabric wrapping being sufficiently permeable to allow application of a liquid or gel to a surface. When applying body wash, for example, the surface is the skin of the bather. When applying a liquid wax, the surface may be that of a vehicle. When applying grease, the surface may comprise mechanical parts of a machine, steel cables, or a metallic surface. When applying a sterilizer of disinfectant, the surface may comprise a frequently touched object such as handles, touchscreens, grab bars, table tops, etc.

Glove 10 includes a pocket 12 stitched on the inside or outside of the permeable fabric where pocket 12 is positioned in or about a palm position of the glove. Other means for securing the bladder may also be provided, such a Velcro or other fasteners known in the art. Glove 10 may also have an inner protective lining (not shown) when applying or dispensing a toxic substance. Pocket 12 is adapted to retain a bladder 14 made of rubber, plastic, polymer. Bladder 14 may even comprise another fabric. Bladder 14 has an internal fluid reservoir surrounded by flexible walls and, in one embodiment, includes plural longitudinal slits 16 on an anterior portion of a wall to pass fluid from the reservoir to an exterior surface of the bladder in order to wash, disinfect, lubricate or treat a surface as needed. Bladder 14 also has a fill port 18 to allow filling of the reservoir with the desired liquid or gel. Fill port 18 is biased in the closed position when filled with liquid or gel but is adapted to open when receiving a puncture probe 20 (FIG. 3a) to fill the bladder with liquid or gel. This may be conventionally achieved by employing a check valve within the fill port. Alternatively, the flexible wall of bladder 14 may bias port 18 in the closed position until forced open by a puncture probe.

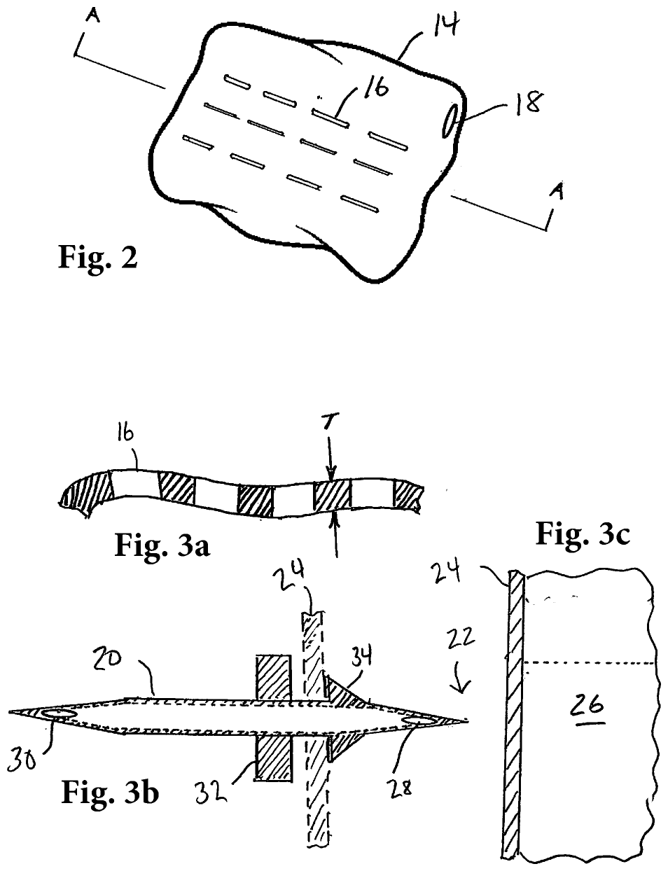

FIG. 2 depicts the bladder 14 in greater detail where slits 16 are shown to have a narrow opening. The width of the openings may range between normally completely closed to normally slightly opened, (perhaps as much as one-eight or greater) when the bladder 14 is unpressurized. The normal width of the opening depends on the viscosity of the liquid or gel to be dispensed where the normal extent of the opening increases with increased viscosity of the liquid or gel being dispensed.

FIG. 3a shows a cross-cut of bladder 14 along line A-A of FIG. 2. As shown in FIG. 3a, the material of walls of bladder 14 have a thickness T and rigidity to retain closure of slits 16 at a less-than-threshold pressure in the reservoir and to allow opening of slits 16 when exceeding that threshold pressure. Pressure in reservoir 14 is applied by a user's hand during dispensing, either by pressing a hand against the surface or by the user squeezing the bladder in the palm or his or her hand. For example, a user may press his or her hand against a surface to increase pressure in bladder 14 to dispense the liquid or gel, or alternatively, the user may squeeze bladder 14 with the palm of his or her hand to increase pressure in bladder 14 to effect dispensing of the liquid or gel.

Thickness T may range from an eight to a about quarter of an inch, more or less, when the bladder is comprised of a material like rubber. Alternatively, bladder 14 may comprise a sack-like plastic pouch having perforations that are sized according to viscosity of the liquid or gel being dispensed. The threshold pressure needed to dispense the liquid or gel is defined by hand pressure and viscosity of the liquid or gel. Fabric of glove 10 may comprise a natural and synthetic fabric and bladder 14 may comprise rubber or flexible plastic, such as polyurethane, polyethylene or polyvinyl chloride. In one embodiment, bladder 14 is rubber having a wall thickness between one-fourth and three-eight inch, more or less. In another embodiment, bladder 14 comprises a perforated polyurethane sack.

FIG. 3b shows an elongated hollow puncture probe 20 that may be used to allow filing of the bladder with a liquid or gel-like substance. Puncture probe 20 has a sharp puncture end 22 that is effective to penetrate a container wall (FIG. 3c) of a substance 26 to be applied to a surface. Substance 26 include body wash, detergents, sterilizers, disinfectants, oils, grease, wax or any other substance that may be applied by hand. In the punctured position shown in FIG. 3b, orifice 28 of probe 20 allows substance 26 to flow through a hollowed journal of probe 20 and exit via orifice 30 into the reservoir of bladder 14 via fill port 18. When using body wash, for example, puncture end 22 punctures the wall of a usually plastic container in which the body wash is purchased by a user. With its cap secured, the container the user may squeeze the container to force the substance into the bladder. Similarly, puncture end 22 may puncture a wax bottle, or an oil can or a grease tube for surface lubrication purposes. Probe 20 includes a stop 32 (shown spaced apart from wall 24, for clarity) and flange portions 34 to retain attachment of the probe 20 to the wall 24 of the substance container. These stops or flanges may circumscribe the cylindrical outer shell of probe 20. As indicated above the substance 26 may comprise body wash, disinfectants, sterilizers, oils, grease or other viscous or gel-like substance. In addition, the portions of the elongated probe may be separated by an elongated tube or small hose to extend the distal ends for more convenient filling of bladder 14.

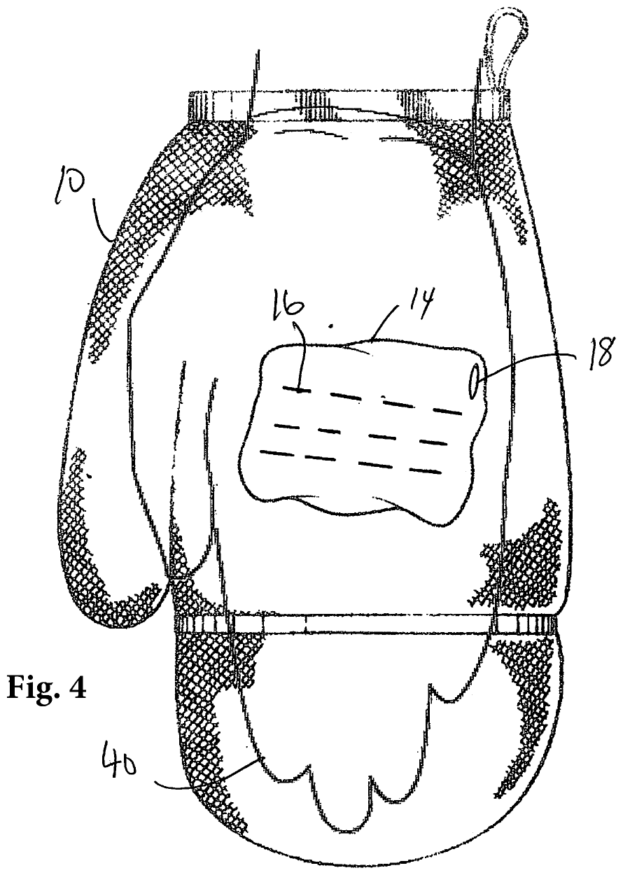

FIG. 4 shows the position of a user's hand inside glove 10 relative to the bladder 14 to permit dispensing by squeezing the bladder to increase pressure within the bladder which, in turn, dispenses the liquid or gel from slits 16. Pressure and consequent dispensing may be accomplished by the user pressing his or her hand against a surface.

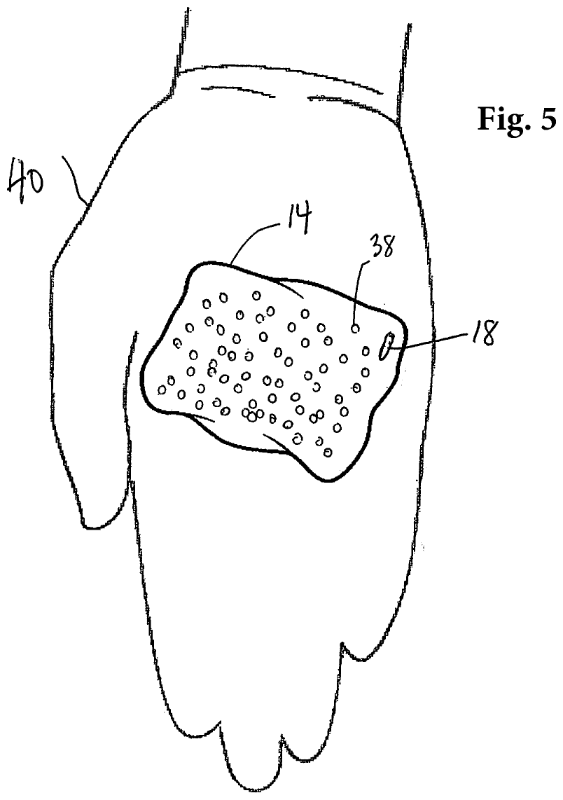

Instead of using slits 16 on bladder 14 that open and close according to internal reservoir pressure, FIG. 5 shows use of a plurality of orifices or holes 38 in bladder 14. Here, bladder 14 includes plural orifices on an anterior wall thereof to allow fluid or gel to flow from the reservoir and ooze it way through the fabric of the applicator glove when under pressure. Here, again, the orifices are sized according to the viscosity of the liquid or gel to be dispensed where larger diameter holes are used of higher viscosity liquids or gels and smaller diameter holes are used for lower viscosity liquids or gels.

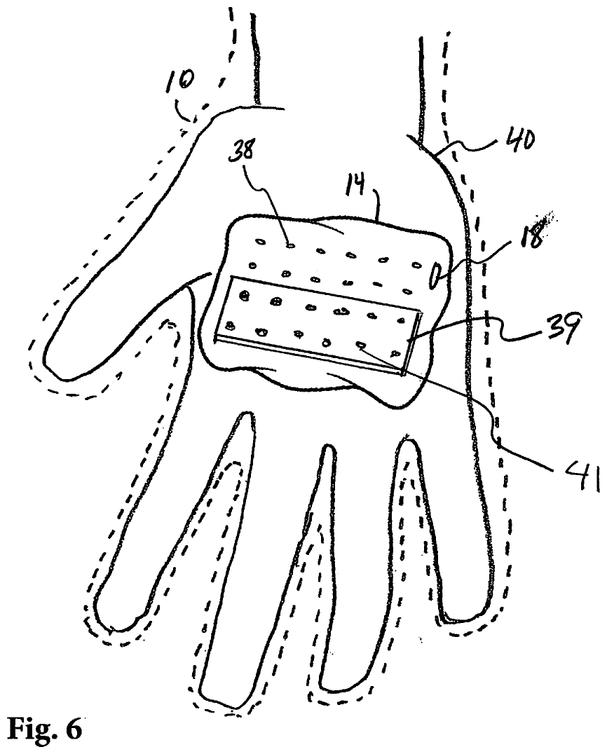

FIG. 6 shows application of the invention using a finger-fit glove 10 for a hand 40 of a user. Similarly, a sack 14 such as a polyurethane sack having a number of orifices 38 is positioned inside a pouch of glove 10 in or near the palm position and adapted for dispensing a liquid or gel upon an increase of pressure inside a reservoir of sack 14. Pressure is increased when the use squeezes the sack or presses his or her hand against a surface upon which the gel or liquid is to be applied. This size or diameter of the orifices 38 are gauged according to the viscosity of the gel or liquid being dispensed and a typical hand pressure applied by a user. The sack 14 may comprise polyurethane, rubber or other flexible material, or even a semipermeable fabric or membrane.

A fill port 18 is also provided to fill or refill the sack 14 with a liquid or gel. What's more, an elongated tubular feed line (not shown), e.g. six feet or more, connected with fill port 18 may be provided to continuously supply liquid of gel to the applicator glove 10 for extended use or applications. Further, sack 14 may be affixed inside or outside the palm position of glove 10 via a stitched pouch on the inside or outside of glove 10, or other attachment means, e.g., Velcro, may be provided between the sack 14 and glove 10 to hold the sack in position during use.

Also shown in FIG. 6 shows another convenient feature of the invention comprising a peel-away segment or tab 39 which, when removed, exposes a set of larger holes 41 thereunder. Those orifices 41 lying under tab 39 are larger while those orifices 38 uncovered by tab 39 are smaller. This way, a single bladder 14 may be adapted for dispensing at least two types of substances according to the desire of the user, who may just peel away or leave intact the tab 39. This feature may be extended to provide three or more peel-away tabs in order to provide an even greater range of viscosities capable of being dispensed by the applicator glove. Alternatively, this feature may be achieved by providing a bladder 14 where one side may have relatively smaller orifices while the other side may have relatively larger orifices. This same arrangement may also be provide with a bladder having slits instead of orifices.

In view of the foregoing, it should be noted that illustrated embodiments described herein are not intended to limit the scope of the invention since various modifications and arrangements may come to those skilled in the art based on the above teachings. As such, the invention includes all such modifications and arrangements embraced by the appended claims.

I claim:

1. An applicator glove useful for applying or dispensing a liquid or gel having a predetermined viscosity, said applicator glove comprising:
(a) a generally liquid permeable fabric wrapping enclosing a hand of a user, said fabric wrapping being sufficiently permeable to allow transfusion of said liquid or gel for application to a surface;
(b) a pocket defined in said permeable fabric positioned in or about a palm position of said glove.
(c) a bladder positioned in said pocket having an internal fluid reservoir surrounded by flexible walls and including plural longitudinal slits on an anterior portion of said bladder that extend from the fluid reservoir to an exterior surface of the bladder to allow fluid flow from the reservoir to the surface,
(d) said bladder having a fill port to allow filling of said reservoir with said liquid or gel, an opening of said fill port being biased in the closed position when filled with liquid or gel but being adapted to receive a puncture probe to fill the bladder with said liquid or gel; and
(e) the walls of said bladder having a thickness and rigidity to retain closure of said slits at less than a reservoir threshold pressure and to allow opening of said slit when exceeding said reservoir threshold pressure, said threshold pressure being defined by hand pressure and viscosity of the liquid or gel in accordance with hand pressure applied by a user when using said glove.

2. The invention of claim 1 wherein said applicator glove comprises one of a natural and synthetic fluid permeable fabric.

3. The invention of claim 1, wherein said bladder comprises rubber, polyurethane, polyethylene or polyvinyl chloride.

4. The invention of claim 3 wherein said bladder has a wall thickness between one-eight and three-eight's of an inch.

5. The invention of claim 1, further comprising a puncture probe to allow filing of said reservoir, wherein said puncture probe is adapted at one end thereof, for insertion into said bladder and, at the other end thereof, for insertion into a container of liquid or gel to be dispensed.

6. The invention of claim 1, wherein said liquid or gel comprises one of a body wash, detergent, disinfectant, sterilizer, wax, grease, and oil.

7. The invention of claim 1, further comprising a tubular fluid line adapted for connection with said fill port to continuously supply fluid or gel to said bladder during use by a user.

8. The invention of claim 1, wherein said bladder includes slits of at least two difference sizes and at least one removable segment adapted to expose slits of a different size according to user desire whereby to enable the bladder to dispense a liquid or gel of a different viscosity.

9. The invention of claim 1, wherein said glove include an inner protective lining to protect the wearer when dispensing a toxic substance.

10. A hand applicator glove useful for dispensing a liquid or gel having a predetermined viscosity, said applicator glove comprising:
(a) a generally liquid permeable fabric wrapping enclosing a hand of a user to define said glove, said fabric wrapping being sufficiently permeable to allow application of said liquid or gel to a surface;
(b) an pocket defined in said permeable fabric positioned in or about a palm position of said glove.
(c) a plastic bladder (polyurethane, polyester) positioned in said pocket having an internal fluid reservoir surrounded by flexible walls and including plural orifices on an anterior wall to allow fluid or gel to flow from the reservoir.

11. The invention of claim 10 wherein said applicator glove comprises one of a natural and synthetic fabric.

12. The invention of claim 10, wherein said bladder comprises rubber, polyurethane, polyethylene, polyvinyl chloride, or plastic.

13. The invention of claim 12 wherein said orifices have a diameter between one-sixteenth and one-fourth inch.

14. The invention of claim 10, further comprising a puncture probe to allow filing if said reservoir, wherein said puncture probe is adapted at one end thereof, for insertion into said bladder and, at the other end thereof, for insertion into a container of liquid or gel to be dispensed.

15. The invention of claim 10, wherein said bladder includes orifices of at least two difference sizes and at least one removable segment adapted to expose orifices of a different size according to user desire whereby to enable the bladder to dispense a liquid or gel of a different viscosity.

16. The invention of claim 10, wherein said glove include an inner protective lining to protect the wearer when dispensing a toxic substance.

17. The invention of claim 10, wherein said liquid or gel comprises one of a body wash, detergent, disinfectant, sterilizer, wax, grease, and oil.
